(19) United States
(12) Patent Application Publication
(10) Pub. No.: US 2025/0269174 A1
(43) Pub. Date: Aug. 28, 2025

(54) MATERIAL REMOVAL DEVICE FOR LEAD EXTRACTION PREPARATION

(71) Applicant: Medtronic, Inc., Minneapolis, MN (US)

(72) Inventors: Karyn C Dobizl, Grant, MN (US); Phillip C. Falkner, Dadeville, AL (US); Claire A. Winchester, Columbia Heights, MN (US); Mark T. Marshall, Cape Coral, FL (US)

(21) Appl. No.: 18/856,785

(22) PCT Filed: Apr. 26, 2023
(86) PCT No.: PCT/US2023/066267

§ 371 Date: Oct. 14, 2024

Related U.S. Application Data
(60) Provisional application No. 63/363,666, filed on Apr. 27, 2022.

(51) Int. Cl. A61N 1/05

(52) U.S. Cl. CPC A61N 1/057; A61N 2001/0578

(57) ABSTRACT

A material removal device includes a housing and a removal element positioned within the housing. The housing defines an opening sized to receive an implantable medical lead of a medical device. The housing is sealed when the implantable medical lead is advanced through the opening. The housing defines a chamber. The removal element is configured to remove material from an outer surface of the implantable medical lead. The chamber is configured to retain the material.

This application claims the benefit of U.S. Provisional Application Ser. No. 63/363,666, filed Apr. 27, 2022, the entire content of which is incorporated herein by reference.

### FIELD

This disclosure generally relates to medical devices and, more particularly, to the extraction of medical devices.

### BACKGROUND

Implantable medical systems commonly include one or more implantable medical leads coupled to an implantable or external medical device to provide a therapy to the patient. As an example, cardiac systems, such as implantable pacemakers, cardioverter-defibrillators, cardiac resynchronization therapy devices and the like, commonly include an IMD such as an implantable pulse generator electrically connected to the heart by at least one implantable medical lead. Electrical stimulation pulses emitted by the IMD travel through the lead and stimulate the heart to deliver a prescribed therapy. Other implantable medical systems, such as neuromodulation stimulators, may have leads implanted in other locations of the patient, e.g., brain, spine, and the like.

In some patients, it may become necessary to extract and replace an implanted lead. For example, a lead may need to be acutely replaced when unacceptable stimulation thresholds are measured during an implant procedure, when the lead fails, or when the endocardial tissue around the lead implantation site becomes infected.

### SUMMARY

In some examples, a material removal device comprises: a housing defining an opening sized to receive an implantable medical lead of a medical device, wherein the housing is sealed when the implantable medical lead is advanced through the opening, and wherein the housing defines a chamber; and a removal element positioned within the housing, wherein the removal element is configured to remove material from an outer surface of the implantable medical lead, and wherein the chamber is configured to retain the material.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an example system including an implantable medical device, in accordance with techniques of this disclosure.

FIG. 2 is a conceptual diagram illustrating an example material removal device, in accordance with techniques of this disclosure.

FIG. 3 is a conceptual diagram illustrating an example implantable medical lead for a medical device, in accordance with techniques of this disclosure.

FIG. 4 is a conceptual diagram illustrating another removal element of an example material removal device, in accordance with techniques of this disclosure.

FIG. 5 is a flow diagram illustrating an example operation for a material removal device, in accordance with techniques of this disclosure.

### DETAILED DESCRIPTION

In general, lead extraction may require preparation of the target lead so that the lead is compatible with a preferred extraction tool, such as a laser sheath, a rotary cutting tool, or a manual dilating sheath. For example, preparation of the lead may ensure that the size of the target lead is compatible with the size of the extraction tool. An example extraction tool is a tubular extraction sheath, which may include a rotating cutting element, a laser, a plasma generator, or an electrocautery system to assist in dissecting tissue around the lead, e.g., fibrotic scar adhesions.

An extraction sheath may define a lumen having an internal diameter sized to allow passage of a lead. In this way, a clinician or other operator may extract the lead through the lumen of the extraction sheath, and thereby remove the lead from the body of the patient. In some examples, the proximal end of the lead may have a relatively bulky connector assembly that plugs into, and makes an electrical connection with, the IMD. Standard connectors that are used to connect leads to pacemakers, such as IS-1, IS-4 and DF-4, have an outside diameter of about 14 French (Fr) to 15 Fr (4.6 mm to 5.0 mm). An inside diameter of an extraction sheath may be about 7 Fr to 13 Fr (2.3 mm to 4.3 mm).

As such, to extract a lead, the lead may be cut near a proximal end of the relatively bulky connector to allow insertion of the lead into the extraction sheath. However, cutting a lead may have one or more disadvantages. For example, once the lead is cut, the conductors of the lead may need to be bound to a suitable tool, e.g., a locking stylet or compression coil, so that traction may be applied to the lead, e.g., to pull it through the extraction sheath lumen and out of the patient. Incorrect preparation of the severed conductors can decrease lead tensile strength and increases the chance of lead breakage. Lead breakage may significantly complicate lead extraction. In addition, some lead designs can lose substantial lead strength when cut, again increasing the chance of lead breakage. Furthermore, cutting the lead to remove the connector may require immobilizing lead insulation and other protective layers overlying the lead conductor with, for example, a ligation suture.

On the other hand, while retaining the connector may maintain the structural strength of the lead, the large disparity between the inner diameter of the extraction sheath and the outside diameter of the lead can cause higher complication rates for vascular damage. For example, in some cases the larger gap between the lead outside diameter and the lead inside diameter can pull the vascular wall into the sheath, which can increase the likelihood of a large vascular tear.

Accordingly, this disclosure is directed to techniques for removing material from an outer surface of a lead to decrease an outer diameter of the lead. For example, a material removal device may be configured to prepare a lead of an IMD by at least partially removing material from the outer surface of components such as a proximal seal and/or a connector attached to the lead. The material removal device may at least partially remove these components by shaving, skiving, slicing, paring, cutting, or the like, to reduce their diameter to a diameter that is compatible with a preferred extraction tool (e.g., to allow insertion of the lead into the extraction sheath without cutting the connecter). In this way, the techniques may allow usage of smaller extraction sheaths, which may advantageously prevent injury to the vasculature of the patient.

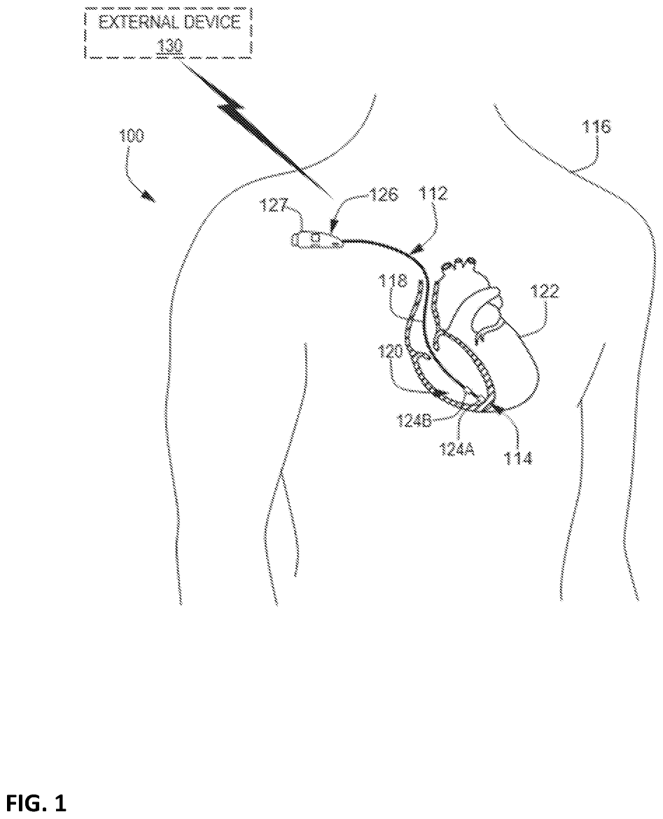

FIG. 1 is a conceptual diagram illustrating a portion of an example implantable medical device system 100. Implantable medical device system 100 may function as a single chamber, e.g., ventricular, pacemaker, as illustrated by the example of FIG. 1 that delivers pacing to a heart 122 of a patient 116. Implantable medical device system 100 may be a temporary or permanent pacemaker. In alternative embodiments, however, implantable medical device system 100 may include one or more leads and function as a multi-chamber pacemaker, such as a dual-chamber or triple chamber pacemaker, that delivers pacing to heart 122 of patient 116. In some examples, the devices and methods of the present disclosure may be implemented for lead preparation of IMDs other than pacemakers, such as defibrillators, cardiac resynchronization therapy (CRT) systems, other electrical stimulator devices, implantable monitors, neurostimulators, and the like.

In the example of FIG. 1, implantable medical device system 100 includes one or more implantable medical leads 112 electrically connected to an IMD 126. Implantable medical lead 112 may include an elongated lead body 118 with a distal portion 120 positioned at a target implantation site 114 within heart 122 such as, for example, a ventricular wall. Lead 112 may be a unipolar, a bipolar, or a multipolar lead, or any type of lead.

A clinician may maneuver distal portion 120 of lead 112 through the vasculature of patient 116 to position distal portion 120 at or near target site 114. For example, the clinician may guide distal portion 120 through the superior vena cava (SVC) to target site 114 on or in a ventricular wall of heart 122, e.g., at the apex of the right ventricle as illustrated in FIG. 1. Implantable medical device system 100 may include a delivery catheter and/or outer member (not shown), and implantable medical lead 112 may be guided and/or maneuvered within a lumen of the delivery catheter in order to approach target site 114.

The implantable medical lead 112 includes one or a plurality of electrodes. In the example of FIG. 1, lead 112 includes electrodes 124A and 124B (collectively, “electrodes 124”) configured to be positioned on, within, or near cardiac tissue at or near target site 114. In some examples, electrodes 124 may provide pacing to heart 122. Electrodes 124 may be electrically connected to conductors (not shown in FIG. 1) extending through lead body 118. In some examples, the conductors are electrically connected to therapy delivery circuitry of IMD 126, with the therapy delivery circuitry configured to provide electrical signals through the conductor to electrodes 124. Electrodes 124 may conduct the electrical signals to the target tissue of heart 122, causing the cardiac muscle, e.g., of the ventricles, to depolarize and, in turn, contract at a regular interval. Electrodes 124 may also be connected to sensing circuitry of IMD 126 via the conductors, and the sensing circuitry may sense activity of heart 122 via electrodes 124.

The electrodes 124 may have various shapes such as tines, helices, screws, rings, partial rings or rings of segments, coils and so on. Again, although a bipolar configuration of lead 112 including two electrodes 124 is illustrated in FIG. 1, in other examples lead 112 may include different numbers of electrodes, such as one electrode, three electrodes, or four electrodes. In configurations in which the lead is a defibrillation lead, the lead may include one or more defibrillation coil electrodes and respective conductors extending through the lead body. In other examples (not shown in FIG. 1), IMD 126 can be connected to two leads (atrium and right ventricle) or three leads (A, RV, LV), or other electrode or lead configurations.

The configuration of the therapy system 100 illustrated in FIG. 1 is merely one example. In other examples, a therapy system may include epicardial leads, subcutaneous, substernal, and/or patch electrodes instead of or in addition to transvenous lead 112 illustrated in FIG. 1. Further, the IMD 126 need not be implanted within the patient 116. That is, although referred to as an implantable medical device, IMD 26 may be an external medical device configured to deliver therapy to patient 116. In examples in which IMD 126 is not implanted in patient 116, IMD 126 may deliver therapies to heart 122 via percutaneous leads that extend through the skin of patient 116 to a variety of positions within or outside of heart 122. In these examples, IMD 126 may additionally or alternatively deliver therapy to another body part of patient 116.

In one or more examples, IMD 126 may include electronic circuitry contained within an enclosure where the circuitry may be configured to deliver cardiac pacing. In the example of FIG. 1, the electronic circuitry within IMD 126 may include therapy delivery circuitry electrically coupled to electrodes 124. The electronic circuitry within IMD 126 may also include sensing circuitry configured to sense electrical activity of heart 122 via electrodes 124. The therapy delivery circuitry may be configured to administer cardiac pacing via electrodes 124, e.g., by delivering pacing pulses in response to expiration of a timer and/or in response to detection of the activity (or absence thereof) of the heart.

In some examples, the system 100 may include an external device 130. For example, external device 130 can be a handheld computing device such as a tablet or a phone, a computer workstation, or a networked computing device. External device 130 can include a user interface that receives input from a clinician, which can include a keypad and a suitable display such as, for example, a touch screen display, or a peripheral pointing device, such as a mouse, via which a user may interact with the user interface. The clinician may also interact with external device 130 remotely via a networked computing device.

After implantable medical lead 112 and electrodes 124 have been temporarily or more permanently implanted in the heart of a patient, lead 112 and electrodes 124 may need to be removed (e.g., due to structural defects, infections, the need to upgrade a pre-existing system, etc.). After implantation for extended periods of time (e.g., greater than about 1 year), leads may develop a dense fibrotic and sometimes calcific process within the thin-walled venous structures or the endocardial surface of the heart or tricuspid valve, which can make lead 112 and electrodes 124 difficult to extract. Complex lead extraction is associated with the risk of vascular injury by traction or perforation, causing tamponade, hemothorax, arteriovenous fistula, tricuspid valve disruption, or possibly pulmonary embolism. Thus, simplifying or otherwise improving lead extraction techniques can have significant value for patient safety.

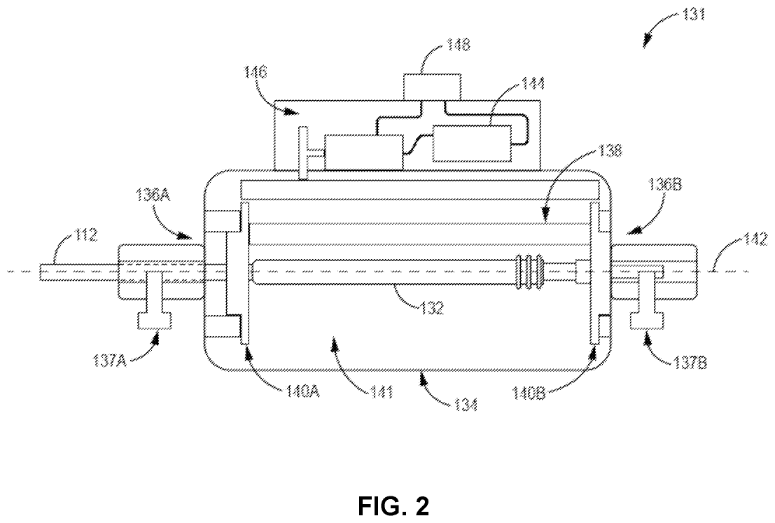

FIG. 2 illustrates a material removal device 131 in accordance with techniques of this disclosure. As described in greater detail below, material removal device 131 may be configured to remove material from an outer surface of lead 112. For example, material removal device 131 may remove material from a connector 132 of lead 112. In this way, material removal device 131 may decrease an outer diameter of lead 112 so that lead 112 is compatible with an extraction sheath or other extraction tool. For example, material removal device 131 may remove material (e.g., a polymer, such as silicone rubber) from connector 132 to decrease an outer diameter of lead 112 so that lead 112 fits within a lumen defined by an extraction sheath.

Material removal device 131 may include a housing 134. Housing 134 may include a casing or cover that encloses and protects internal components of material removal device 131. Housing 134 may also protect the user from injury by components of material removal device 131 instead housing, and may also contain material removed from a lead, e.g., to avoid contamination.

Housing 134 may be formed of one or more materials such as plastic, metal, etc. Housing 134 may define one or more openings, such as openings 136A-136B, that are sized to receive lead 112. When lead is advanced through openings 136 of housing 134, housing 134 may be sealed. For example, with the exception of openings 136, housing 134 may be sealed to prevent ingress (e.g., of fluid) and egress (e.g., of material removed from lead 112). Housing 134 may include one or more locking elements, such as locking elements 137A and 137B (collectively, “locking elements 137”), configured to secure lead 112 relative to housing 134. For example, locking elements 137 may be spring-loaded locks configured to secure lead 112 so that lead 112 is rotationally fixed (and in some cases also longitudinally fixed) relative to housing 134.

Material removal device 131 may include a removal element 138 configured to remove material from an outer surface of lead 112. Removal element 138 may be positioned withing housing 134. Removal element 138 may remove material from the outer surface of lead 112 by cutting, shaving, slicing, etc. In some examples, removal element 138 may be a blade (e.g., a skiver blade). Removal element 138 may be formed from a durable material, such as steel, to ensure removal element 138 remains sharp and effective. Various shapes of removal element 138 are contemplated by this disclosure. That said, removal element 138 may be as long or longer than connector 132 to ensure that material from the entire outer surface of connector 132 is removed.

Removal element 138 may be mechanically coupled to one or more rotary bearings, such as rotary bearings 140A and 140B (collectively, “rotary bearings 140”). Rotary bearings 140 may be configured to rotate relative to housing 134, thereby facilitating rotation of removal element 138 around lead 112 (which is rotationally fixed relative to housing 134). As removal element 138 rotates around lead 112, removal element 138 may glide along the outer surface of lead 112, shaving off layers from lead 112 (e.g., connector 132) until the desired outer diameter is achieved. Housing 134 may retain the removed material (e.g., to reduce the risk of incision contamination) within a chamber 141 defined by housing 134 until a clinician or other operator opens housing 134 (e.g., housing 134 may be configured to be opened and closed to allow for the safe discarding of removed material). In some examples, removal element 138 may be spring-loaded so that removal element 138 is biased towards a longitudinal axis 142, in this way ensuring that removal element 138 is contacting and thus shaving lead 112. Additionally or alternatively, removal element 138 may be cam-adjusted.

In some examples, material removal device 131 may be manually operated. For example, material removal device 131 may include a crank, a lever, or a handle configured to actuate removal element 138 to remove the material from the outer surface of lead 112 (e.g., facilitate manual rotation of rotary bearings 140). In other examples, material removal device 131 may be electrically operated. For example, material removal device 131 may include a battery 144 that actuates a gearbox 146 (both of which may be positioned within housing 134). Battery 144 and gearbox 146 may be configured to actuate removal element 138 to remove the material from the outer surface of lead 112. For example, gearbox 146 may transmit power and motion to rotary bearings 140 to rotate removal element 138 around lead 112. In examples where material removal device 131 is electrically operated, material removal device 131 may include an actuation element 148 (e.g., a switch, a button, etc.) that causes battery 144 to actuate gearbox 146. A wiper on one or both ends of material removal device 131 may be used to wipe off removed material from lead 112 as lead 112 is removed from material removal device 131.

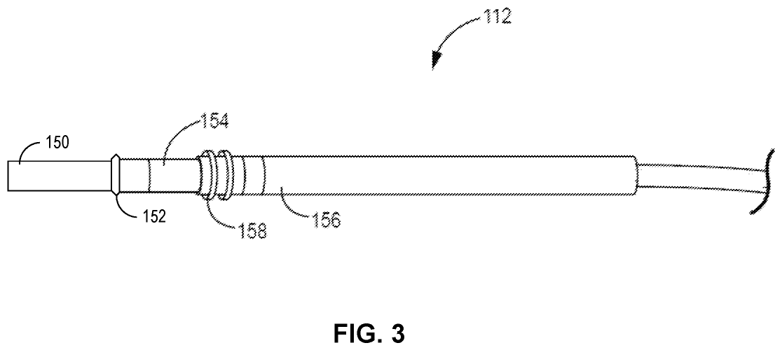

FIG. 3 illustrates in more detail lead 112. As shown in FIG. 3, lead 112 may include a terminal pin contact 150, one or more seals 152 (made, e.g., of a polymer such as silicone rubber), a ring electrode 154, and a connector 156 having a grip sleeve 158 (made, e.g., of a polymer such as silicone rubber). The connector may be connected to a proximal end of a lead (e.g., lead 112) that has a distal end with a distal electrode (not shown in FIG. 3). The distal electrode may be coupled to terminal pin contact 150 via an elongate insulated conductor, and a second electrode on the distal end may be coupled to ring electrode 154 via another elongated conductor. Connector 156 may be configured for connection, for example, to a pulse generator device, which may be implanted in a subcutaneous pocket in the pectoral region of a patient's chest. To utilize a smaller diameter extraction sheath for an extraction of lead 112, a material removal device as described herein may be used by advancing it over terminal pin contact 150 and operating the removal element to at least partially remove material (e.g., the silicone rubber of the seals and grip sleeve of the lead assembly) from lead 112.

The material removal device may provide significant value to physicians in terms of faster lead preparation time for extraction, overall reduced procedure times, consistent lead mechanics during extraction, reduction of risk of injury to patients and physicians, and reduction of risk of incision contamination.

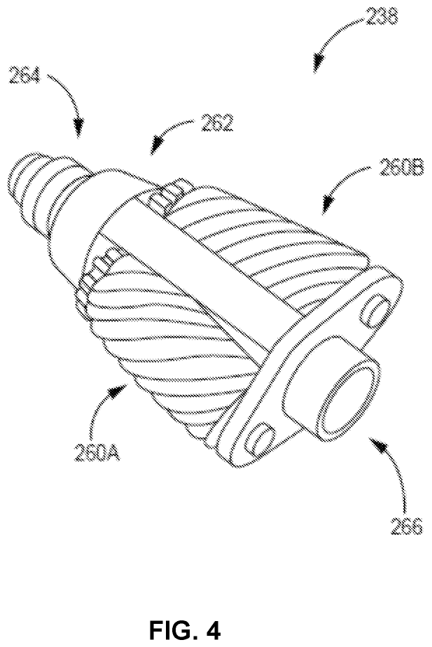

FIG. 4 illustrates another example of a removal element 238 of a material removal device. Although not shown, removal element 238 may be rotationally fixed relative to a housing of a material removal device. Removal element 238 may include a rotating drum skiver configured to remove material from an outer surface of a lead. Removal element 238 may include one or more drums, such as drums 260A-260B that rotate about their respective axes. Each of drums 260 may have a sharp outer surface (e.g., may define a blade) configured to remove material from an outer surface of a lead. In some examples, drums 260 may be spring-loaded to allow an appropriate application of pressure on a lead, irrespective of the size and/or design of the lead.

Removal element 238 may include one or more gears 262 configured to transmit power and motion to rotate drums 260. Gears 262 may be operated manually (e.g., using a crank, a lever, a handle, etc.) or electrically (e.g., using an electric motor). In any case, removal element 238 may remove the outer layer of a lead in a substantially continuous process until a desired outer diameter of the lead is achieved.

In some examples, removal element 238 may include a proximal stabilizing element 264 and a distal stabilizing element 266. Both proximal stabilizing element 264 and distal stabilizing element 266 may be configured to stabilize and support a lead positioned within removal element 238. In some examples, proximal stabilizing element 264 may include a connector pin spring-loaded collet configured to hold a proximal portion of a lead. In some examples, distal stabilizing element 266 may include a spring-loaded stabilizer configured to support a distal portion of a lead. Both proximal stabilizing element 264 and distal stabilizing element 266 may be configured to reduce rotation of removal element 238 as a whole relative to the lead. A wiper on one or both ends of removal element 238 may be used to wipe off the lead as the lead is removed from removal element 238.

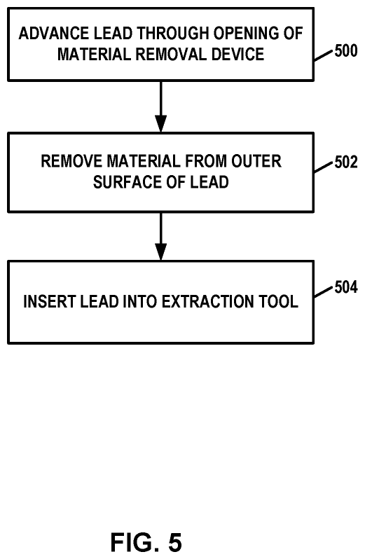

FIG. 5 is a flow diagram illustrating an example operation of a material removal device. Although the example operation of FIG. 5 is described as being performed by material removal device 131 of FIG. 2, in other examples some or all of the example operations may be performed by another material removal device.

In some examples, lead 112 may need to be removed (e.g., because lead 112 is no longer functioning correctly, because lead 112 needs to be replaced, etc.). To extract lead 112, a clinician may use specialized tools, such as an extraction sheath, to detach lead 112 from any surrounding tissues or organs and remove lead 112 from patient 116.

In accordance with techniques of this disclosure, a clinician may advance lead 112 through an opening, such as opening 136A, of housing 134 of material removal device 131 (500). In some examples, housing 134 may define only one opening, and in other examples housing 134 may define two or more openings (e.g., opening 136A and opening 136B). In any case, when lead is advanced through the one or more openings 136 of housing 134, housing 134 may be sealed. Locking elements 137 may secure lead 112 relative to housing 134. For example, locking elements 137 may secure lead 112 so that lead 112 is rotationally fixed (and in some cases also longitudinally fixed) relative to housing 134.

Material removal device 131 may use removal element 138 to remove material from an outer surface of lead 112 (502). Removal element 138 may be positioned withing housing 134. Removal element 138 may remove material from the outer surface of lead 112 by cutting, shaving, slicing, etc. In some examples, removal element 138 may be a blade (e.g., a skiver blade). Removal element 138 may be as long or longer than connector 132 to ensure that material from the entire outer surface of connector 132 is removed.

In some examples, removal element 138 may be mechanically coupled to rotary bearings 140 that facilitate rotation of removal element 138 around lead 112 (which is rotationally fixed relative to housing 134). As removal element 138 rotates around lead 112, removal element 138 may glide along the outer surface of lead 112, shaving off layers from lead 112 (e.g., connector 132) until the desired outer diameter is achieved. Housing 134 may retain the removed material (e.g., to reduce the risk of incision contamination) within a chamber 141 defined by housing 134 until a clinician or other operator opens housing 134. In some examples, material removal device 131 may be manually operated. In other examples, material removal device 131 may be electrically operated.

Material removal device 131 may remove enough material from lead 112 to render lead 112 compatible with a preferred extraction tool, such as an extraction sheath with a relatively small inner diameter. For example, after removing enough material from an outer diameter of lead 112 so that lead 112 fits within a lumen defined by an extraction tool, a clinician may insert at least a portion of lead 112 into the lumen of the extraction tool (504). The clinician may then operate the extraction tool to extract lead 112 from patient 116.

This disclosure includes various examples, such as the following examples.

Example 1: A material removal device includes a housing defining an opening sized to receive an implantable medical lead of a medical device, wherein the housing is sealed when the implantable medical lead is advanced through the opening, and wherein the housing defines a chamber; and a removal element positioned within the housing, wherein the removal element is configured to remove material from an outer surface of the implantable medical lead, and wherein the chamber is configured to retain the material.

Example 2: The material removal device of example 1, wherein the material removal device further includes one or more rotary bearings configured to facilitate rotation of the removal element around the implantable medical lead.

Example 3: The material removal device of example 1 or 2, wherein the material removal device further includes one or more locking elements configured to secure the implantable medical lead so that the implantable medical lead is rotationally fixed relative to the housing.

Example 4: The material removal device of any one or more of examples 1 to 3, wherein the removal element is as long or longer than a connector of the lead.

Example 5: The material removal device of any one or more of examples 1 to 4, wherein the material removal device further includes one or more of a crank, a lever, or a handle configured to actuate the removal element to remove the material from the outer surface of the implantable medical lead.

Example 6: The material removal device of any one or more of examples 1 to 4, wherein the material removal device further includes a battery and a gearbox, wherein the battery and the gearbox are positioned within the housing and configured to actuate the removal element to remove the material from the outer surface of the implantable medical lead.

Example 7: The material removal device of any one or more of examples 1 to 6, wherein the removal element is biased towards a longitudinal axis of the material removal device.

Example 8: The material removal device of any one or more of examples 3 to 7, wherein the removal element includes one or more drums having an outer surface configured to remove the material from the outer surface of the implantable medical lead.

Example 9: The material removal device of example 8, wherein the one or drums are spring-loaded.

Example 10: The material removal device of example 8 or 9, wherein the removal element further includes one or more gears configured to transmit power and motion to rotate the one or more drums.

Example 11: The material removal device of any one or more of examples 8 to 10, wherein the removal element further includes a proximal stabilizing element configured to stabilize and support the implantable medical lead.

Example 12: The material removal device of example 11, wherein the proximal stabilizing element includes a connector pin spring-loaded collet.

Example 13: The material removal device of any one or more of examples 8 to 12, wherein the removal element further includes a distal stabilizing element configured to stabilize and support the implantable medical lead.

Example 14: The material removal device of example 13, wherein the proximal stabilizing element includes a spring-loaded stabilizer.

Example 15: The material removal device of any one or more of examples 1 to 14, wherein the housing is configured to be opened and closed.

Example 16: The material removal device of any one or more of examples 1 to 15, wherein the opening is a first opening, and wherein the housing further defines a second opening sized to receive the implantable medical lead, wherein the housing is sealed when the implantable medical lead is advanced through the first opening and the second opening.

Example 17: A method includes advancing an implantable medical lead of a medical device through an opening of a housing of a material removal device, wherein the housing is sealed when the implantable medical lead is advanced through the opening, and wherein the housing defines a chamber; removing, by a removal element of the material removal device positioned within the housing, material from an outer surface of the implantable medical lead, wherein the chamber is configured to retain the material; and inserting at least a portion of the implantable medical lead into a lumen of an extraction sheath.

Example 18: The method of example 17, further including rotating, by one or more rotary bearings, the removal element around the implantable medical lead.

Example 19: The method of example 17 or 18, further including securing, by one or more locking elements, the implantable medical lead so that the implantable medical lead is rotationally fixed relative to the housing.

Example 20: The method of any one or more of examples 17 to 19, wherein the removal element is as long or longer than a connector of the lead.

Various embodiments of the invention have been described. These and other embodiments are within the scope of the following claims.

1. A material removal device comprising:
a housing defining an opening sized to receive an implantable medical lead of a medical device, wherein the housing is sealed when the implantable medical lead is advanced through the opening, and wherein the housing defines a chamber; and
a removal element positioned within the housing, wherein the removal element is configured to remove material from an outer surface of the implantable medical lead, and wherein the chamber is configured to retain the material.

2. The material removal device of claim 1, wherein the material removal device further comprises one or more rotary bearings configured to facilitate rotation of the removal element around the implantable medical lead.

3. The material removal device of claim 1, wherein the material removal device further comprises one or more locking elements configured to secure the implantable medical lead so that the implantable medical lead is rotationally fixed relative to the housing.

4. The material removal device of claim 1, wherein the removal element is as long or longer than a connector of the lead.

5. The material removal device of claim 1, wherein the material removal device further comprises one or more of a crank, a lever, or a handle configured to actuate the removal element to remove the material from the outer surface of the implantable medical lead.

6. The material removal device of claim 1, wherein the material removal device further comprises a battery and a gearbox, wherein the battery and the gearbox are positioned within the housing and configured to actuate the removal element to remove the material from the outer surface of the implantable medical lead.

7. The material removal device of claim 1, wherein the removal element is biased towards a longitudinal axis of the material removal device.

8. The material removal device of claim 3, wherein the removal element comprises one or more drums having an outer surface configured to remove the material from the outer surface of the implantable medical lead.

9. The material removal device of claim 8, wherein the one or drums are spring-loaded.

10. The material removal device of claim 8, wherein the removal element further comprises one or more gears configured to transmit power and motion to rotate the one or more drums.

11. The material removal device of claim 8, wherein the removal element further comprises a proximal stabilizing element configured to stabilize and support the implantable medical lead.

12. The material removal device of claim 11, wherein the proximal stabilizing element comprises a connector pin spring-loaded collet.

13. The material removal device of claim 8, wherein the removal element further comprises a distal stabilizing element configured to stabilize and support the implantable medical lead.

14. The material removal device of claim 13, wherein the proximal stabilizing element comprises a spring-loaded stabilizer.

15. The material removal device of claim 1, wherein the housing is configured to be opened and closed.

16. The material removal device of claim 1, wherein the opening is a first opening, and wherein the housing further defines a second opening sized to receive the implantable medical lead, wherein the housing is sealed when the implantable medical lead is advanced through the first opening and the second opening.

17. A method comprising:
advancing an implantable medical lead of a medical device through an opening of a housing of a material removal device, wherein the housing is sealed when the implantable medical device is advanced through the opening, and wherein the housing defines a chamber;
removing, by a removal element of the material removal device positioned within the housing, material from an outer surface of the implantable medical lead, wherein the chamber is configured to retain the material; and
inserting at least a portion of the implantable medical lead into a lumen of an extraction sheath.

18. The method of claim 17, further comprising rotating, by one or more rotary bearings, the removal element around the implantable medical lead.

19. The method of claim 17, further comprising securing, by one or more locking elements, the implantable medical lead so that the implantable medical lead is rotationally fixed relative to the housing.

20. The method of claim 17, wherein the removal element is as long or longer than a connector of the lead.
